(19) United States
(12) Patent Application Publication
(10) Pub. No.: US 2024/0216676 A1
(43) Pub. Date: Jul. 4, 2024

(54) HIGH-FREQUENCY ENERGY DELIVERY DEVICE WITH PRECISE TEMPERATURE TRACKING

(71) Applicant: WON TECH Co., Ltd., Daejeon (KR)

(72) Inventors: Jong Won KIM, Daejeon (KR); Young Seok SEO, Sejong-si (KR); Young Sik KIM, Daejeon (KR); Se Jong KIM, Daejeon (KR)

(21) Appl. No.: 17/921,874

(22) PCT Filed: Oct. 5, 2022
(86) PCT No.: PCT/KR2022/014950

§ 371 Date: Oct. 27, 2022

(30) Foreign Application Priority Data
Dec. 16, 2021 (KR) 10-2021-0180328

(51) Int. Cl. A61N 1/06; A61B 5/01; A61N 1/08; A61N 1/40

(52) U.S. Cl. CPC A61N 1/06; A61B 5/01; A61N 1/08; A61N 1/403

(57) ABSTRACT

Disclosed herein is a high-frequency energy transmission device which noninvasively transmits high-frequency energy to the deep layer of the skin. The high-frequency energy transmission device includes: a tip; and a handpiece mechanically and electrically combined with the tip. The tip includes: an electrode for noninvasively emitting high-frequency energy to the deep layer of the skin; and a plurality of temperature sensors arranged in such a way that the electrode is interposed therebetween.

### TECHNICAL FIELD

The present invention relates to a high-frequency energy transmission device, and more specifically, to a high-frequency energy transmission device which measures temperature of the skin touching an electrode before or after irradiation of high-frequency energy, and measures temperature of the center of the electrode more accurately so as to measure the rise of skin temperature according to the transmission of high-frequency energy, thereby preventing skin burn.

### BACKGROUND ART

A high-frequency energy transmission device is a device having an electrode for transmitting high-frequency energy (radio frequency energy) to the skin. The high-frequency energy transmission device stimulates collagen by transmitting high-frequency energy to the tissue layer under the skin through various types of high-frequency electrodes, such as monopolar electrodes, bipolar electrodes, multi-polar electrodes, and the like, thereby treating the skin for skin contraction or lifting, wrinkle alleviation, and other cosmetic purposes.

A domestic skin care device can perform a simple procedure by transmitting energy of a lower frequency output. However, a high-frequency energy transmission device used as a medical device outputs frequencies of 6 Mhz to 7 Mhz to more effectively perform a skin care procedure. Unlike the domestic device, the high-frequency energy transmission device is used just by an expert operator who holds qualification, such as a doctor.

The high-frequency energy transmission device has effect of heating human tissues. Among such high-frequency energy transmission devices, the high-frequency energy transmission device as a skin care device has a purpose for heating a deep collagen layer below the skin. However, in a case in which heat generated by heating stays on the surface of the skin as it is, burn may occur. So, in order to prevent such burn, it is necessary to perform cooling.

Moreover, since a pain as well as burn may be caused due to heating in transmitting high-frequency energy, the recent high-frequency energy transmission device has vibration in a vertical direction (back-and-forth) or in a horizontal direction (up-and-down or right-and-left) in order to reduce the pain.

In order to maximize the convenience of a procedure by preventing burns or pains on the skin surface, it is necessary to precisely measure temperature of the electrode of the high-frequency energy transmission device and temperature of the skin touching the electrode, but it is difficult to measure the temperature at one point since the high-frequency energy is non-focused.

In addition, since the high-frequency electrode of the high-frequency energy transmission device touches the skin in the form of a surface electrode rather than a point electrode, it is difficult to specify the temperature of the electrode.

### DISCLOSURE

### Technical Problem

Accordingly, the present invention has been made in view of the above-mentioned problems occurring in the related art, and it is an object of the present invention to provide a high-frequency energy transmission device which includes at least two temperature sensors and a high-frequency electrode disposed between the at least two temperature sensors to measure temperature of the surface of a specific range at positions where the temperature sensors are respectively disposed and to store the measured temperature value, thereby measuring temperature of the skin touching the electrode in real time through the temperature sensors.

The aspects of the present disclosure are not limited to those mentioned above, and other aspects not mentioned herein will be clearly understood by those skilled in the art from the following description.

### Technical Solution

To accomplish the above-mentioned objects, according to the present invention, there is provided a high-frequency energy transmission device which noninvasively transmits high-frequency energy to the deep layer of the skin, including: a tip; and a handpiece mechanically and electrically combined with the tip, wherein the tip comprises: an electrode for noninvasively emitting high-frequency energy to the deep layer of the skin; and a plurality of temperature sensors arranged in such a way that the electrode is interposed therebetween.

In an embodiment of the present invention, the high-frequency energy transmission device further includes a control unit electrically connected with the handpiece. The tip further includes: a first electrode of which the width is longer than the length; and a plurality of first electrode temperature sensors disposed at an upper portion and a lower portion of the first electrode, wherein the first electrode temperature sensors are formed at the upper portion and the lower portion of the first electrode in pairs to have the shortest distance from one another, are arranged to be adjacent to the first electrode, and senses a temperature value of the skin surface getting in contact with the first electrode temperature sensors to transmit the sensed temperature value to the control unit.

In another embodiment of the present invention, the high-frequency energy transmission device further includes a control unit electrically connected with the handpiece. The tip further includes: a second electrode of which the width and the length are the same; and a plurality of second electrode temperature sensors disposed at edges of the second electrode, wherein the second electrode temperature sensors are arranged to be adjacent to the second electrode, and senses a temperature value of the skin surface getting in contact with the second electrode temperature sensors to transmit the sensed temperature value to the control unit.

In an embodiment of the present invention, the high-frequency energy transmission device further includes a memory electrically connected with the control unit. The control unit receives temperature values that the first electrode temperature sensors or the second electrode temperature sensors sense while getting in contact with the skin, calculates an average value, designates the average value as a central temperature value of the electrode, blocks transmission of the high-frequency energy or stops the operation of the handpiece in a case in which the average value exceeds a preset value, and stores the temperature value or the average value in the memory.

### Advantageous Effects

According to an embodiment of the present invention, the high-frequency energy transmission device includes at least two temperature sensors and a high-frequency electrode disposed between the at least two temperature sensors to measure temperature of the surface of a specific range at positions where the temperature sensors are respectively disposed and to store the measured temperature value, thereby measuring temperature of the skin touching the electrode in real time through the temperature sensors.

The advantages of the present disclosure are not limited to the above-mentioned advantages, and other advantages, which are not specifically mentioned herein, will be clearly understood by those skilled in the art from the following description.

### DESCRIPTION OF DRAWINGS

FIG. 1 is a schematic diagram of a high-frequency energy transmission device according to an embodiment of the present invention.

FIG. 2 is a diagram of the high-frequency energy transmission device according to the embodiment of the present invention.

FIG. 3 is a diagram of a tip of the high-frequency energy transmission device according to the embodiment of the present invention.

FIG. 4 is a diagram illustrating a state in which high-frequency energy is transmitted from the tip of the high-frequency energy transmission device to the deep layer of the skin according to the embodiment of the present invention.

FIG. 5 is a view illustrating an electrode and temperature sensors of the high-frequency energy transmission device according to the embodiment of the present invention.

FIG. 6 is a view illustrating an electrode and temperature sensors of a high-frequency energy transmission device according to another embodiment of the present invention.

FIG. 7 is a view illustrating an electrode and temperature sensors of a high-frequency energy transmission device according to another embodiment of the present invention.

### MODE FOR INVENTION

Hereinafter, the present invention will be described in detail with reference to the accompanying drawings. However, embodiments of the present invention may be implemented in several different forms and are not limited to the embodiments described herein. In addition, parts irrelevant to description are omitted in the drawings in order to clearly explain embodiments of the present invention. Similar parts are denoted by similar reference numerals throughout this specification.

Throughout this specification, when a part is referred to as being “connected” to another part, this includes “direct connection” and “indirect connection” via an intervening part. Also, when a certain part “includes” a certain component, other components are not excluded unless explicitly described otherwise, and other components may in fact be included.

The terms used in the following description are intended to merely describe specific embodiments, but not intended to limit the invention. An expression of the singular number includes an expression of the plural number, so long as it is clearly read differently. The terms such as “include” and “have” are intended to indicate that features, numbers, steps, operations, elements, components, or combinations thereof used in the following description exist and it should thus be understood that the possibility of existence or addition of one or more other different features, numbers, steps, operations, elements, components, or combinations thereof is not excluded.

Hereinafter, embodiments of the present invention will be described in detail with reference to the accompanying drawings.

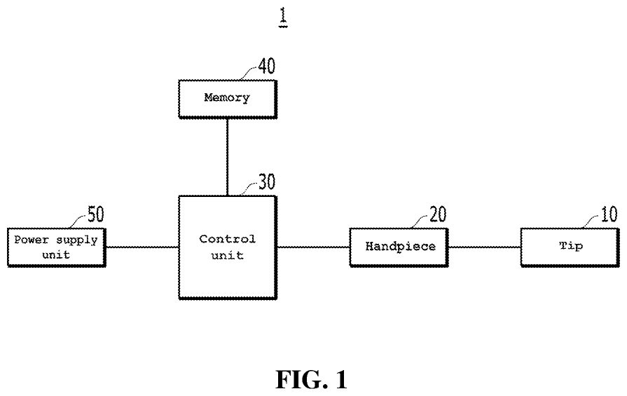

FIG. 1 is a schematic diagram of a high-frequency energy transmission device according to an embodiment of the present invention. Referring to FIG. 1, a high-frequency energy transmission device 1 is connected to a power supply unit 50 in a wired or wireless manner around a control unit 30, and is supplied with power through the connection.

The high-frequency energy transmission device 1 includes a memory 40, and the memory 40 is connected to the control unit 30 in a wired or wireless manner. The high-frequency energy transmission device 1 includes a handpiece 20, and the handpiece 20 is connected to the control unit 30 in a wired or wireless manner.

A tip 10 is fit to the handpiece 20, and the handpiece 20 includes a terminal inserted to transmit energy to the tip 10. When the handpiece 20 and the tip 10 are connected to each other, refrigerant can flow therebetween, and a flow path for the flow of the refrigerant is formed.

The control unit 30 transmits high-frequency energy to the handpiece 20, and the transmitted high-frequency energy is transmitted to the tip 10. The control unit 30 can block the high-frequency energy transmitted to the handpiece 20 and the tip 10, and can stop the operation of the handpiece 20.

The control unit 30 transmits high-frequency energy having preset power and frequency to the handpiece 20.

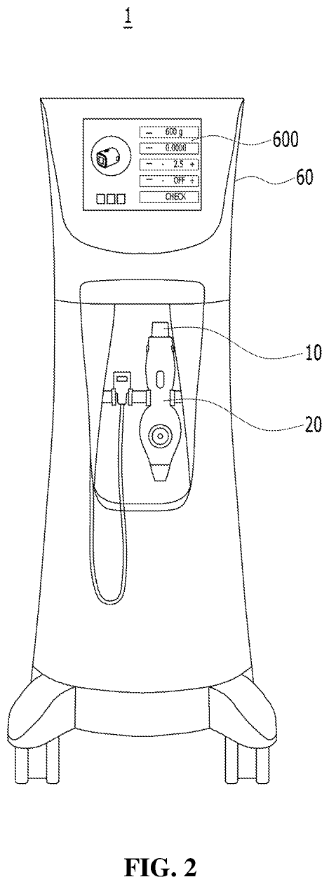

FIG. 2 is a diagram of the high-frequency energy transmission device according to the embodiment of the present invention. Referring to FIG. 2, the high-frequency energy transmission device 1 includes a movable body 60 and a display 600.

The high-frequency energy transmission device 1 includes a holding slot capable of holding the handpiece 20. The high-frequency energy transmission device 1 includes the display 600 for visual display. The display 600 visually displays the type of the tip 10, and visually displays contents performed through the handpiece 20.

The handpiece 20 is formed in an ergonomically flexible shape so that a user can hold it with the hand, and includes a button disposed at a portion thereof to turn on and off power of the high-frequency energy or to control transmission of the high-frequency energy. The handpiece 20 further includes an adjustment button for adjusting output intensity of the high-frequency energy and the number of shots of the high-frequency energy.

The display 600 of the high-frequency energy transmission device 1 may be attached to a main body 60, or may have a height-adjustable structure capable of being separated from the main body 60.

A moving wheel of the high-frequency energy transmission device 1 is provided with a stabilizer to prevent the main body 60 from falling down. The display 600 of the high-frequency energy transmission device 1 visually displays warning information for preventing the main body 60 from falling down.

The display 600 of the high-frequency energy transmission device 1 not only displays the contents of operation performed through the handpiece 20 but also has a touch screen function. Accordingly, the display 600 includes an interface capable of adjusting the degree of operation of the handpiece 20.

The handpiece 20 is connected to the main body 60 in a wired manner to receive high-frequency energy and refrigerant, and is connected to the control unit 30 for controlling the handpiece 20 in a wired or wireless manner to be automatically controlled.

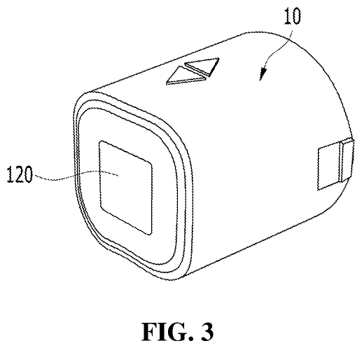

FIG. 3 is a diagram of a tip of the high-frequency energy transmission device according to the embodiment of the present invention. Referring to FIG. 3, the tip 10 has a first electrode 100 formed on the front surface thereof in a rectangular shape, and the first electrode 100 is formed at a portion of the center of the front surface of the tip.

A peripheral portion of the first electrode 100 may be electrically insulated, and the high-frequency energy is emitted only through the first electrode 100 of the rectangular shape.

The tip 10 has a circuit capable of receiving high-frequency energy from the handpiece 20, and the circuit is connected to the first electrode 100 to transmit high-frequency energy.

The tip 10 has a coupling portion capable of being coupled to the handpiece 20, and the coupling portion has a flow path through which the refrigerant is transferred from a refrigerant flow path of the handpiece 20 to the coupling portion.

The tip 10 has a flow path capable of transferring the refrigerant supplied from the handpiece 20 to the first electrode 100. The refrigerant may be sprayed around the first electrode 100 in the form of gas, or may be periodically sprayed in the form of a pulse for a predetermined time.

The first electrode 100 is formed in a rectangular shape. Furthermore, the first electrode 100 may be formed in a size of 4 cm² to transmit high-frequency energy to a wide skin surface. Alternatively, the first electrode 100 may be formed in a size of 0.25 cm² to transmit high-frequency energy around the eyes.

The first electrode 100 may be formed in a rectangular shape, of which the width is longer than the length.

The tip 10 may be varied in structure according to the shape and size of the first electrode 100. In a case in which the first electrode 100 is formed in a size of 4 cm², the tip 10 is formed as a trapezoidal pillar similar to a square pillar.

In a case in which the first electrode 100 is formed in a size of 0.25 cm², the tip 10 is formed as a trapezoidal pillar similar to a quadrangular pyramid.

In a case in which the first electrode 100 is formed in a rectangular shape, the tip 10 may have a structure corresponding to the shape of the first electrode 100.

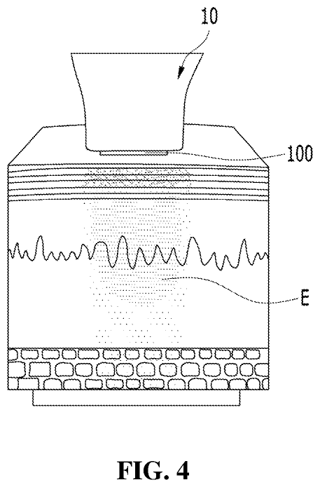

FIG. 4 is a diagram illustrating a state in which high-frequency energy is transmitted from the tip of the high-frequency energy transmission device to the deep layer of the skin according to the embodiment of the present invention. Referring to FIG. 4, high-frequency energy (E) is emitted through the tip 10 and the first electrode 100 to heat the deep layer of the skin.

The high-frequency alternating current emitted from the first electrode 100 flows to a grounding pad to rapidly convert the polarity of molecules of the subcutaneous tissues, so that the molecular level generates vibration and friction to generate heat in the tissue core.

The heated tissues may be a dermal layer between the epidermal layer and the subcutaneous tissue, and may enhance the elasticity by reinforcing collagen fiber by thermal energy.

In order to prevent the heat generated from the first electrode 100 from causing burn on the skin surface, refrigerant may be sprayed from the tip 10 adjacent to the first electrode 100. The refrigerant is sprayed in the form of a pulse in order to prevent burn of the skin surface and reduce pains, and can cool the heat generated by the high-frequency energy of the first electrode 100.

The high-frequency energy emitted from the first electrode 100 may be a pulse type irradiated for a predetermined period of time at a predetermined interval. The first electrode 100 is controlled by the control unit 30, and irradiates high-frequency energy in a different way from the pulse period of the refrigerant to effectively stimulate the collagen layer of the core of the skin tissue.

The irradiation period of the high-frequency energy emitted from the first electrode 100 and the pulse period of the refrigerant are different from each other in emission period of time, and the irradiation period of the high-frequency energy may be longer or shorter than the emission period of the refrigerant.

The irradiation period of the high-frequency energy emitted from the first electrode 100 and the pulse period of the refrigerant may be shown in turn or may be overlapped. The high-frequency energy may be irradiated from the first electrode 100 after the refrigerant pulse is emitted. Contrariwise, the refrigerant pulse is emitted after the high-frequency energy is irradiated from the first electrode 100.

In order to minimize the distance that the high-frequency alternating current emitted from the first electrode is transmitted to the grounding pad, the grounding pad is attached on a human body.

In order to minutely control output of the high-frequency alternating current emitted from the first electrode 100 by the control unit 30, a measuring unit for measuring impedance of the skin surface is disposed.

The impedance measured through the first electrode 100 and the measuring unit may be utilized as data for outputting high-frequency energy by the control unit 30, and the measured value of the impedance is stored in a memory 40.

The front surface of the first electrode 100 is coated with a dielectric material to transmit the high-frequency energy while getting in contact with the skin. The dielectric material can transmit high-frequency energy by electrically capacitively combining the skin and the first electrode 100.

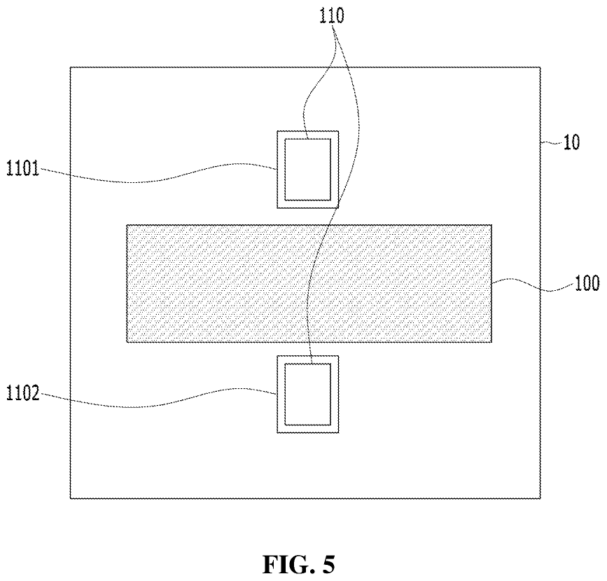

FIG. 5 is a view illustrating the electrode and temperature sensors of the high-frequency energy transmission device according to the embodiment of the present invention. Referring to FIG. 5, the first electrode 100 may be formed to have the width longer than the length, or be formed on the front surface of the tip 10 in a rectangular shape which is horizontally long.

A first electrode temperature sensor 110 is formed around the first electrode 100, and includes a first temperature sensor 1101 formed on an upper portion thereof, and a second temperature sensor 1102 formed on a lower portion of the first electrode 100 in a pair with the first temperature sensor 1101. The first temperature sensor 1101 and the second temperature sensor 1102 are formed to be the closest to the first electrode 100.

Even if a dielectric material is applied to the front surface of the first electrode 100, the first temperature sensor 1101 and the second temperature sensor 1102 can be arranged to be the closest to the first electrode 100. In a case in which the first electrode 100 comes into contact with the skin, the first temperature sensor 1101 and the second temperature sensor 1102 measure the temperature of the skin getting in contact with the first electrode 100.

The first electrode temperature sensor 110 measures temperature in real time whenever the first electrode 100 comes into contact with the skin and calculates a temperature value. The plurality of first electrode temperature sensors 110 correspond to each other, and can calculate an average value by receiving the measured temperature values from the control unit 30.

The first temperature sensor 1101 corresponds to the second temperature sensor 1102, and transmits the measured temperature value to the control unit 30 to calculate an average value. The calculated average value is designated as a temperature value at the center of the first electrode 100.

In a case in which the calculated average value exceeds a preset threshold temperature value, the control unit 30 controls the handpiece 20 or the first electrode 100 or blocks the high-frequency energy in order to prevent the high-frequency energy from being transmitted from the first electrode 100.

In a case in which the calculated average value is less than a preset threshold temperature value, the control unit 30 transmits the high-frequency energy to the first electrode 100 as a preset control value.

The plurality of first electrode temperature sensors 110 are formed around the first electrode 100 in two or more pairs.

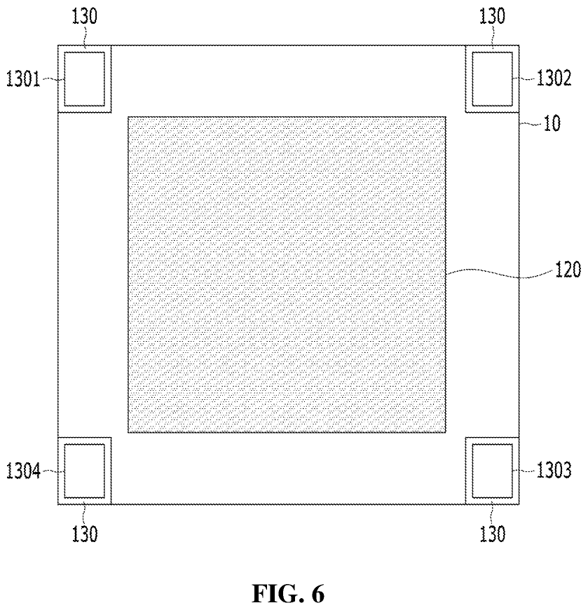

FIG. 6 is a view illustrating an electrode and a temperature sensor of a high-frequency energy transmission device according to another embodiment of the present invention. Referring to FIG. 6, a second electrode 120 is formed in a square shape of which the width and the length are equal.

Second electrode temperature sensors 130 are formed around the second electrode 120, namely, are disposed at corners of the square second electrode 120. The second electrode temperature sensors 130 include a third temperature sensor 1301 disposed at the top corner of the left side of the second electrode 120, and a fifth temperature sensor 1303 disposed at the bottom corner of the right side in a pair with the third temperature sensor 1301. Additionally, a fourth temperature sensor 1302 is disposed at the top corner of the right side of the second electrode 120, and a sixth temperature sensor 1304 disposed at the bottom corner of the left side in a pair with the fourth temperature sensor 1302.

a right lower corner by being paired with the third temperature sensor 1301. In addition, a fourth temperature sensor 1302 may be disposed at a right upper edge, and a sixth temperature sensor 1304 may be disposed at a lower left corner by being paired with the fourth temperature sensor.

Even if a dielectric material is applied to the front surface of the second electrode 120, the third temperature sensor 1301, the fourth temperature sensor 1302, the fifth temperature sensor 1303, and the sixth temperature sensor 1304 are arranged to be the closest to the second electrode 120. In a case in which the second electrode 120 comes into contact with the skin, the third temperature sensor 1301, the fourth temperature sensor 1302, the fifth temperature sensor 1303, and the sixth temperature sensor 1304 can measure temperature of the skin getting in contact with the second electrode 120.

The second electrode temperature sensor 130 measures temperature in real time whenever the second electrode 120 comes into contact with the skin and calculates a temperature value. The plurality of second electrode temperature sensors 130 correspond to each other, and can calculate an average value by receiving the measured temperature values from the control unit 30.

The third temperature sensor 1301 corresponds to the fifth temperature sensor 1103, the fourth temperature sensor 1302 corresponds to the sixth temperature sensor 1104. The third to sixth temperature sensors transmit the measured temperature values to the control unit 30 to calculate an average value. The calculated average value is designated as a temperature value at the center of the second electrode 120.

The temperature values measured through the third temperature sensor 1301 and the fifth temperature sensor 1303 are calculated to obtain an average value, and the temperature values measured through the fourth temperature sensor 1302 and the sixth temperature sensor 1304 are calculated to obtain an average value.

In a case in which the calculated average value exceeds a preset threshold temperature value, the control unit 30 can control the handpiece 20 or the second electrode 120 or block high-frequency energy from being transmitted from the second electrode 120.

In a case in which the calculated average value is less than the preset threshold temperature value, the control unit 30 transmits the high-frequency energy to the second electrode 120 as a preset control value.

The second electrode temperature sensors 130 are arranged to have the shortest distance from one another, and are arranged at the top, bottom, left, and right of the second electrode 120.

The plurality of second electrode temperature sensors 130 are formed around the second electrode 120 in three or more pairs.

The first electrode temperature sensors 110 or the second electrode temperature sensors 130 sense the temperature of the skin while getting in contact with the skin, and continuously transmit the sensed temperature values to the control unit 30. The sensed temperature values are transmitted to the control unit 30, and at the same time, stored in the memory 40.

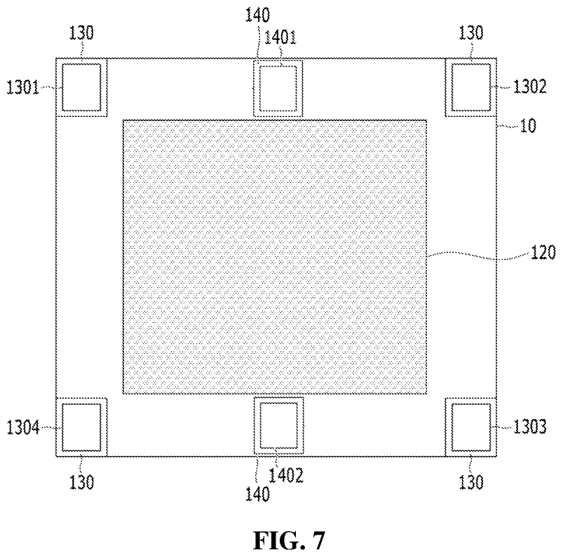

FIG. 7 is a view illustrating an electrode and a temperature sensor of a high-frequency energy transmission device according to a further embodiment of the present invention. Referring to FIG. 7, the high-frequency energy transmission device includes a second electrode 120, second electrode temperature sensors 130, and second electrode central temperature sensors 140 respectively disposed at an upper portion and a lower portion of the second electrode 120.

The second electrode central temperature sensors 140 are disposed on a central line in a vertical direction with respect to the second electrode 120 in a pair to correspond to each other with respect to the second electrode 120. A seventh temperature sensor 1401 is disposed at the upper portion of the second electrode 120, and an eighth temperature sensor 1402 is disposed at the lower portion of the second electrode 120.

An average value is calculated by using temperature values measured through the seventh temperature sensor 1401 and the eighth temperature sensor 1402, and the calculated average value is designated as a central temperature value of the second electrode 120.

The second electrode temperature sensors 130 respectively disposed at right and left sides with respect to the second electrode central temperature sensor 140 detect a temperature value for an edge portion of the second electrode 120.

In order to measure an edge temperature value of the second electrode 120, the third temperature sensor 1301 corresponds to the sixth temperature sensor 1304, the fourth temperature sensor 1302 corresponds to the fifth temperature sensor 1303. An average value is calculated by using the temperature values detected from the corresponding temperature sensors.

The third temperature sensor 1301 and the sixth temperature sensor 1304 calculate an average value from the detected temperature values, and the fourth temperature sensor 1302 and the fifth temperature sensor 1303 calculate an average value from the detected temperature values. The average value of the detected temperature values is designated as an average temperature value for the edge portion of the second electrode 120.

The average value of the temperature values detected through the third temperature sensor 1301 and the sixth temperature sensor 1304 is designated as an average temperature value for the left edge portion of the second electrode 120, and the average value of the temperature values detected through the fourth temperature sensor 1302 and the fifth temperature sensor 1303 is designated as an average temperature value for the right edge portion of the second electrode 120.

The central temperature value calculated through the second electrode central temperature sensor 140, and the average temperature values of the left and right edge portions calculated through the second electrode central temperature sensors 140 are compared with each other.

In a case in which the average temperature value of the edge portion is greater than the central temperature value and is equal to or greater than a preset threshold temperature value, the control unit 30 recognizes it as a strange phenomenon that high-frequency energy is unequally distributed to the edge portion, and controls the handpiece 20 or the second electrode 120 to prevent the high-frequency energy from being transmitted from the second electrode 120 or blocks the high-frequency energy.

The foregoing description of the present disclosure has been presented for the purposes of illustration and description. It is apparent to a person having ordinary skill in the art to which the present disclosure relates that the present disclosure can be easily modified into other detailed forms without changing the technical principle or essential features of the present disclosure.

Therefore, the foregoing embodiments should be regarded as illustrative rather than limiting in all aspects. In an example, each component which has been described as a unitary part can be implemented as distributed parts. Similarly, each component which has been described as distributed parts can also be implemented as a combined part.

While the present disclosure has been shown and described with reference to various embodiments thereof, it will be understood by those skilled in the art that various changes in form and details may be made therein without departing from the spirit and scope of the present disclosure as defined by the appended claims and their equivalents.

### INDUSTRIAL APPLICABILITY

The present invention is applicable to a high-frequency energy transmission device which is industrially applicable.

1. A high-frequency energy transmission device which noninvasively transmits high-frequency energy to the deep layer of the skin, comprising:
a tip; and
a handpiece mechanically and electrically combined with the tip,
wherein the tip comprises: an electrode for noninvasively emitting high-frequency energy to the deep layer of the skin; and
a plurality of temperature sensors arranged in such a way that the electrode is interposed therebetween.

2. The high-frequency energy transmission device according to claim 1, further comprising:
a control unit electrically connected with the handpiece,
wherein the tip further comprises:
a first electrode of which the width is longer than the length; and
a plurality of first electrode temperature sensors disposed at an upper portion and a lower portion of the first electrode, and
wherein the first electrode temperature sensors are formed at the upper portion and the lower portion of the first electrode in pairs to have the shortest distance from one another, are arranged to be adjacent to the first electrode, and senses a temperature value of the skin surface getting in contact with the first electrode temperature sensors to transmit the sensed temperature value to the control unit.

3. The high-frequency energy transmission device according to claim 1, further comprising:
a control unit electrically connected with the handpiece,
wherein the tip further comprises:
a second electrode of which the width and the length are the same; and
a plurality of second electrode temperature sensors disposed at edges of the second electrode, and
wherein the second electrode temperature sensors are arranged to be adjacent to the second electrode, and senses a temperature value of the skin surface getting in contact with the second electrode temperature sensors to transmit the sensed temperature value to the control unit.

4. The high-frequency energy transmission device according to claim 2, further comprising:
a memory electrically connected with the control unit,
wherein the control unit receives temperature values that the first electrode temperature sensors or the second electrode temperature sensors sense while getting in contact with the skin, calculates an average value, designates the average value as a central temperature value of the electrode, blocks transmission of the high-frequency energy or stops the operation of the handpiece in a case in which the average value exceeds a preset value, and stores the temperature value or the average value in the memory.
